(19) United States
(12) Patent Application Publication
(10) Pub. No.: US 2006/0235370 A1
(43) Pub. Date: Oct. 19, 2006

(54) Method of regulating mammalian keratinous tissue

(76) Inventors: John Erich Oblong, Loveland, OH (US); Shekhar Mitra, Indian Hill, OH (US); Helen Rochelle Kemp, Hamilton, OH (US); Mark David Evans, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/363,002

(22) Filed: Feb. 27, 2006

Related U.S. Application Data
(60) Provisional application No. 60/667,930, filed on Apr. 4, 2005.
(60) Provisional application No. 60/692,641, filed on Jun. 21, 2005.
(60) Provisional application No. 60/694,758, filed on Jun. 28, 2005.

(51) Int. Cl. A61B 18/18

(52) U.S. Cl. 606/9

(57) ABSTRACT

Method for providing a benefit to mammalian keratinous tissue, comprising applying a composition comprising one or more hair growth regulating compounds to the keratinous tissue and delivering energy to the keratinous tissue by means of an energy delivery device.

### CROSS REFERENCE TO RELATED APPLICATION

This application claims the benefit of U.S. Provisional Application No. 60/667,930, filed Apr. 4, 2005, U.S. Provisional Application No. 60/692,641, filed Jun. 21, 2005 and U.S. Provisional Application No. 60/694,758, filed Jun. 28, 2005.

### FIELD OF THE INVENTION

The present invention relates to methods for providing a benefit to mammalian keratinous tissue, including regulating hair growth, comprising the steps of applying a composition comprising one or more hair growth regulating compounds and delivering energy to the keratinous tissue.

### BACKGROUND OF THE INVENTION

Hair growth regulation often is desired to improve one's appearance and hygiene. Various methods and personal care products have been developed to remove unwanted hair, for example, shaving, electrolysis, waxing and depilatory creams. However, these methods have several drawbacks. Shaving offers only short term hair removal, and must be repeated. Electrolysis and waxing can keep a treated area free of hair for prolonged periods of time, but can be expensive and/or painful. It is therefore desirable to develop methods of regulating hair growth that reduce or eliminate the need for repeated hair removal and the drawbacks associated therewith.

Energy delivery devices such as lasers, diode lasers and flash lamps can be used to regulate hair growth. Traditionally, these devices have been expensive, cumbersome, and suitable for use only by professionals. In addition, these devices may cause undesirable damage to cells and tissues surrounding the hair follicle. The development of smaller, hand-held energy delivery devices overcomes many of these drawbacks; however, smaller devices may result in less efficient and shorter-lasting hair removal. There exists a continuing need, therefore, to provide more effective and efficient methods of regulating unwanted hair growth.

Applicants believe that more effective regulation of hair growth may be achieved by applying compounds useful for regulating hair growth either simultaneously or sequentially with delivery of energy to the keratinous tissue. Various compounds, for example, hexamidine, panthenol, pantothenic acid derivatives, BHT, green tea extracts, and catechin compounds to name a few, are known to provide effective regulation of hair growth, including hair appearance, and bodily malodor. Without being limited by theory, it is believed that hair growth regulating compounds inhibit protease activity in and surrounding the hair follicle, making it difficult for new hair to take root and grow. Additionally, these compounds can inhibit specific cellular signaling events, for example, those mediated via vascular endothelial growth factor (VEGF). It is further believed that energy delivered through the skin and hair is transmitted into the hair bulb region, where it is preferentially absorbed by existing melanin. The absorbed energy is in turn emitted as thermal energy into the surrounding follicular region, where it raises the temperature sufficiently to result in protein denaturation and inhibition of cell proliferation. This contributes to a reduction in the rate of growth of existing hair and a reduction in generation of new hair.

Applicants further believe that spin traps may be useful in modulating hair growth by impacting several key biochemical processes related to hair growth, such as reducing mRNA expression levels of inducible nitric oxide synthase (iNOS), matrix metalloproteases (MMPs), and cyclooxygenase-2 (COX-2). Spin traps also have been found to inhibit iNOS and COX-2 enzymatic activity directly. Without being limited by theory, it is believed that because nitric oxide (NO) plays a key signal transduction role in stimulating follicular cell growth, as well as in providing a signal for stimulating and maintaining angiogenesis, reducing iNOS enzyme levels may reduce the amount of NO being produced in follicular cells. This in turn may reduce hair growth rates, due to a lower vasculature system that is essential for the anagen phase of hair follicles. MMPs are key components in restructuring the extracellular matrix during follicular progression through the dermis of skin during early anagen phase. Additionally, MMPs play a role in angiogenesis, a key process for vascularization of the hair follicle during early anagen phase, as well as in maintaining the vasculature bed during the entire anagen phase. Thus, it is believed that inhibition of MMPs also will lead to a reduction in hair growth rates. Finally, since prostaglandins play a critical role in regulating cellular proliferation, it is believed that inhibition of COX-2 will lead to a reduced proliferative index of hair follicles cells, ultimately leading to decreased hair growth rates.

Additional hair growth regulators may include glucans, for example alpha- and beta-glucans, and compounds known as free radical spin traps (herein, “spin traps”). Glucans are thought to provide a number of benefits to keratinous tissue, including improved moisturization, sustained hydration, collagen synthesis, wound-healing properties, and may act as antioxidants and immune system stimulants. Without being limited by theory, it is believed that alpha- and beta-glucans act on fibroblasts to stimulate procollagen synthesis. Fibroblasts have glucan receptors, and produce procollagen in the dermis of the skin. Procollagen is converted into collagen; therefore, procollagen synthesis helps strengthen the skin matrix and improve the appearance of aging skin. On the surface of keratinous tissue, glucans can form a thin film that moisturizes and protects. In addition, although glucans exhibit anti-oxidant capabilities, this is not known to occur to a significant extent in hair follicle cells. Therefore, glucans may have an adverse impact on hair growth, and may be useful for inhibiting or otherwise regulating hair growth. In summary, glucans are thought to provide regulation of keratinous tissue by both direct and indirect mechanisms. Alpha- and beta-glucans are found in a variety of sources, including grains such as oats, and typically are large molecules. However, large molecules, including certain beta-glucans, tend to exhibit limited ability to penetrate into the deeper layers of keratinous tissue. There also exists a need, therefore, to increase the penetration of large molecules into the skin to reach hair follicle cells.

### SUMMARY OF THE INVENTION

The present invention meets the aforementioned need of providing a benefit to mammalian keratinous tissue, which may directly or indirectly result from regulation of unwanted hair growth. Applicants believe that the combination of hair growth regulating compounds with delivery of energy to the skin will result in an additive and/or synergistic effect, and thus provide more efficient regulation of hair growth, of bodily malodor, and also may increase the penetration of large molecules into the skin to reach hair follicle cells. The energy may be in a variety of forms, including, but not limited to, light, heat, sound (including ultrasonic waves), electrical energy, magnetic energy, electromagnetic energy (including radio frequency energy), and combinations thereof.

The following represent some non-limiting embodiments of the present invention.

According to a first embodiment of the present invention, a method for providing a benefit to mammalian keratinous tissue is provided, comprising the steps of applying a composition comprising one or more hair growth regulating compounds, and delivering energy to the keratinous tissue by means of an energy delivery device. The compounds may be applied simultaneously and/or sequentially with the energy.

According to yet another embodiment of the present invention, a kit is provided for providing a benefit to mammalian keratinous tissue, comprising a composition, which in turn comprises one or more hair growth regulating compounds, and an energy delivery device.

According to yet another embodiment of the present invention, an application regimen is provided for providing a benefit to mammalian keratinous tissue, comprising a period of time during which energy is delivered to the keratinous tissue and a period of time during which a composition is applied to the keratinous tissue. The composition comprises one or more hair growth regulating compounds.

These and other aspects and advantages of the present invention will become evident to those skilled in the art from a reading of the following detailed description of the invention.

### DETAILED DESCRIPTION OF THE INVENTION

Whereas the specification concludes with claims that particularly point out and distinctly claim the present invention, it is believed that the invention will be better understood from the following details.

The present invention describes a method for providing a benefit to mammalian keratinous tissue, comprising the steps of applying a composition comprising one or more hair growth regulating compounds and delivering energy to the keratinous tissue by means of an energy delivery device. Benefits include, but are not limited to, regulation of hair growth, of bodily malodor, of the appearance of skin and/or of the appearance of hair. The present invention further comprises a wide variety of application regimens, and a kit suitable, for example, for facilitating compliance with an application regimen.

The energy delivery device may deliver energy in a variety of forms, including but not limited to light, heat, sound (including ultrasonic waves), electrical energy, magnetic energy, electromagnetic energy (including radiofrequency waves and microwaves), and combinations thereof. The light energy may be delivered by devices including, but not limited to, lasers, diode lasers, diode laser bars, diode laser arrays, flash lamps, intense pulsed light (IPL) sources, and combinations thereof. The energy delivery device optionally may include a means for heating and/or cooling the skin prior to, simultaneously with, or after delivery of energy, and may include a means for storing compositions and for delivering one or more compositions through the device. In one embodiment, the device is hand held. In another embodiment, the device is cordless.

The compositions of the present invention comprise one or more hair growth regulating compounds. The compositions of the present invention may take a variety of final forms, non-limiting examples of which include lotions, creams, liquids, gels, emulsions, multi-phase emulsions, and solid forms, and may be applied to the skin via a variety of means. The compositions of the present invention optionally may include additional skin care actives useful for regulating the condition of mammalian skin, skin conditioning agents, perfumes and deodorizers.

Each of the above and additional elements is described herein.

In all embodiments of the present invention, all percentages are by weight of the total composition, unless specifically stated otherwise. All ratios are weight ratios, unless specifically stated otherwise. All ranges are inclusive and combinable. The number of significant digits conveys neither limitations on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified. .

Herein, “energy delivery device,” means any device used to deliver energy to the skin, hair and other keratinous tissue. Herein, “delivery of energy,” means that the surface of the keratinous tissue is exposed to the energy emanating from the energy delivery device, where it may penetrate to the desired layers of the tissue, including the hair shaft and/or hair follicle.

Herein, “energy” includes but is not limited to energy in the form of light, heat, sound (including ultrasonic waves), electrical energy, magnetic energy, electromagnetic energy (including radiofrequency waves and microwaves), and combinations thereof.

Herein, “continuous level” means that the energy delivered by the device, or energy output, remains at an essentially constant level between the time of device activation and the time of device deactivation.

Herein, “pulsed” means that between the time of device activation and the time of device deactivation, the energy output varies in a predictable manner, characterized by periods of higher output (pulses) alternating with periods of lower output. The onset of pulses typically is sudden as opposed to gradual. “Predictable” means that the pulse peak intensities, pulse shapes, pulse durations, and the temporal spacings between the pulses are substantially identical. The duration of the pulses and the time between pulses may range from on the order of femtoseconds (or 10-15 seconds) to seconds.

Herein, “modulated” means that between the time of device activation and the time of device deactivation, the energy output is characterized by periods of higher output (pulses) alternating with periods of lower output, wherein the pulse intensities, pulse shapes, pulse durations, and the temporal spacings between the pulses may vary significantly. The variation is dependent upon conditions of use, for example the thickness of the skin, hair density, hair coloration, hair thickness, etc. The duration of the pulses and the time between pulses may range from on the order of tenths of a second to tens of seconds.

Herein, “non-modulated” means that between the time of device activation and the time of device deactivation, the energy output is essentially constant, but may be reduced or eliminated under certain circumstances. When the energy output is reduced or eliminated, the device remains activated. Circumstances affecting energy output typically are not directly under control of the operator, but are the result of parameters either programmed or built into the device. These circumstances may include, but are not limited to, lack of contact of the device with the skin, or feedback to the device indicating that a given area of skin already has been treated.

Herein, “hand-held,” as used in reference to an energy delivery device, means that the device is of a weight and dimensions suitable for an average adult human to comfortably hold.

Herein, “cordless” means that the device may be operated without being connected to an electrical outlet by means of an electrical cord.

Herein, “regulating hair growth” means reducing, modulating, inhibiting, attenuating, retarding, and/or diminishing hair growth, and further may include regulating hair appearance. Herein, “hair growth regulating compound” means a compound useful for regulating the growth of hair, and is understood not to encompass compounds such as depilatories. “Hair,” as used herein, includes hair on any part of the body. Regulating hair growth is demonstrated by reducing the frequency of hair removal, or by the tactile and/or visual perception of hair reduction. “Regulating hair appearance,” as used herein, means that the hair on the skin is visually perceived to be softer, finer, or less noticeable. Additionally, one may notice positive changes in the ease, frequency, and efficacy of hair removal. Reduction and/or inhibition of hair growth may be indicated qualitatively or quantitatively. Qualitative indications of reduction and/or inhibition of hair growth include a person's perception of, for example, softer, finer, or less noticeable hair. Quantitative indications of reduction and/or inhibition of hair growth include, for example, when the mass of the hair removed by shaving is reduced.

It is to be understood that hair growth regulating compounds may impart other benefits to the skin and/or keratinous tissue, that may result directly or indirectly from regulation of hair growth, or that may be independent of hair growth regulation, for example, improving skin appearance, for example, skin rejuvenation, reducing the appearance of fine lines, wrinkles, enlarged pores, shine, etc.; improving skin feel;

regulating chronic skin conditions such as acne and seborrhea, and regulating bodily malodor.

Herein, “bodily malodor” refers to an unpleasant or otherwise offensive odor emanating from the body of a mammal, and alternatively of a human being. Bodily malodor may originate from biochemical processes and/or result directly or indirectly from the application of energy. “Regulating bodily malodor” means reducing, inhibiting, attenuating, masking or eliminating bodily malodor. “Keratinous tissue,” as used herein, means keratin-containing layers disposed as the outermost protective covering of mammals and includes, but is not limited to, skin and hair. “Skin,” as used herein, means all layers of the skin, including the epidermal, dermal and subcutaneous layers. “Topical application,” as used herein, means to apply or spread a composition onto the surface of the keratinous tissue to which energy is to be delivered, or has been delivered. “Dermatologically-acceptable,” as used herein, means that the compositions or components thereof so described are suitable for use in contact with mammalian keratinous tissue without undue toxicity, incompatibility, instability, allergic response, and the like, is compatible with the actives of the present invention and with the energy delivery device, and will not cause undue safety or toxicity concerns.

Herein, “orally acceptable” means that the compositions or components thereof so described are suitable for oral ingestion by a mammal without undue toxicity, incompatibility, instability, allergic response, and the like.

The phrase “treating an/the expanse of skin with an acute hair growth technology,” as used herein, includes shaving, epilating, contacting the skin with a depilatory, waxing, and the like.

“Simultaneously,” as used herein, means that the application of the compositions to the skin and the delivery of energy to the skin essentially are part of a single, continuous action, or alternatively, that the elapsed time between application of the compositions to the skin and the delivery of energy to the skin is minimal. Herein, “minimal” is understood to comprise approximately five minutes or less. An example of minimal elapsed time would comprise the time required to apply the composition to the area of skin requiring application, after which energy would be delivered without further significant delay.

“Sequential” or “sequentially,” as used herein, means that the application of the compositions to the skin occurs prior to and/or after delivery of energy to the skin. Herein, “prior to” means that the first topical application of the compositions to a given area of skin occurs, followed by a period of at least five minutes, and alternatively at least 10 minutes, during which no delivery of energy to the skin occurs, followed by a period during which energy is appropriately delivered to the area of skin to which the composition has been applied. Herein, “after” means that energy is appropriately delivered to a given area of skin, followed by a period of at least five minutes, and alternatively at least 10 minutes, during which no delivery of energy to the skin occurs, followed by application of the composition to essentially the same area of skin to which energy has been applied.

It is to be understood that “sequentially” encompasses application regimens that alternate application of the compositions to the skin and delivery of energy. The ratio of the number of applications of the composition to the number of times energy is delivered may vary widely, and is within the discretion of those skilled in the art. For example, the compositions may be applied repeatedly and regularly, and energy delivered to the skin less frequently. One example of sequential application would be an application regimen in which the composition is repeatedly administered on a daily or weekly basis, and delivery of energy occurs on a monthly basis.

Herein, “coverage indicator” means any means of indicating to the user that energy has been applied to an area of keratinous tissue. The indication may be in response to a stimulus resulting from, for example, a color change, a chemical change, a change in the level of energy, a change in temperature, etc. Examples of indicators include, but are not limited to, compositions containing a color indicator, an electronic sensor that may emit an auditory or visual signal, or any combination thereof.

Herein, “dietary supplement” means an orally acceptable, dietary ingredient intended to supplement a regular diet, non-limiting examples of which include, vitamins, minerals, herbs or other botanicals, amino acids, enzymes and metabolites. It is to be understood “dietary supplement” includes orally ingestible alpha and/or beta-glucans, wherein the orally-ingestible form comprises a greater concentration of glucans than are found in the unaltered or natural substance from which the glucans are derived. The form in which the dietary supplement is administered may vary widely, and includes, for example, tablets, capsules, gel tablets, and liquids, and may be incorporated into a foodstuff or beverage.

I. Energy

The energy delivered to the keratinous tissue includes, but is not limited to, energy in the form of light, heat, sound (including ultrasonic waves), electrical energy, magnetic energy, electromagnetic energy (including radio frequency energy), and combinations thereof. In one embodiment, the energy is delivered to the keratinous tissue in the form of light, heat, and ultrasonic sources, and combinations thereof. In yet another embodiment, the energy is delivered in the form of light, and alternatively, laser-generated light.

The energy may be delivered from these various sources in a manner that is continuous, pulsed, modulated, non-modulated, and combinations thereof. Alternatively, the energy is non-modulated.

A. Light energy sources

“Light energy,” as used herein, means light emitted from laser and/or non-laser light sources. The light energy may be coherent or non-coherent, monochromatic or polychromatic, and collimated, diffuse or divergent. Polychromatic light may be filtered to provide the desired wavelength or a selected band of wavelengths.

Laser light sources include solid-state lasers, gaseous lasers and combinations thereof. Non-limiting examples of solid-state laser light sources include Nd:YAG (Neodymium:Yttrium Aluminum Garnet), ruby and alexandrite. Non-limiting examples of gaseous laser sources include helium-neon, argon, and carbon dioxide. Examples of the use of suitable laser light sources are disclosed in U.S. Pat. Nos. 6,063,074 and 6,152,917, both issued to Tankovich. Additional laser light sources include, but are not limited to, diode lasers, diode laser bars, or diode laser arrays. See, for example, U.S. Pat. No. 6,273,885, issued to Koop et al.

Non-limiting examples of non-laser light sources include flashlamps, halogen lamps, light-emitting diodes (LED's), intense pulsed light (IPL) sources and combinations thereof. The wavelengths may comprise the ultraviolet, visible, near-infrared and infrared regions of the electromagnetic spectrum. Alternatively, the wavelength will be in the visible light range. Examples of suitable non-laser light sources are disclosed in U.S. Pat. Nos. Tb 5,885,273, 6,174,325, and 6,280,438, all issued to Eckhouse et al.

In one embodiment, the energy delivery device is of a weight and dimensions suitable for an average adult human to hold comfortably in one hand. Alternatively, the volume of the device is 1500 cubic centimeters (ccm) or less, alternatively the volume is less than 1000 ccm, and alternatively, the volume is less than 750 ccm. Alternatively, the weight of the device is 1 kilogram (kg) or less, alternatively the weight is less than 750 grams, and alternatively the weight is less than 500 grams. An example of a suitable hand-held energy delivery device is described in US 2004/0167499 Al (Grove et al.).

The energy delivery device further may comprise a means for pre-heating or for cooling the skin. See, for example, U.S. Pat. No. 6,273,884, issued to Altshuler et al. Additionally or alternatively, the energy delivery device is cordless.

B. Heat energy sources

The energy delivered to the keratinous tissue may be in the form of heat energy. The keratinous tissue, including portions of the hair within the follicles, may be heated by broad band radiation emitted by the heat source, for example, a flash lamp. Alternatively, the heat may be generated by means of visible radiation, delivered, for example, by a high intensity lamp such as a xenon arc lamp. The heat delivery device may include a means for preventing overheating of the skin, for example, by manually or automatically distancing the apparatus from the skin, by pumping air into region surrounding the skin selected time after the flashing of the flash lamp, or by other suitable means of cooling the skin. Examples of devices that utilize heat energy are disclosed in U.S. Pat. No. 6,187,001, issued to Azar et al.

C. Ultrasound energy sources

The energy delivered to the keratinous tissue may be in the form of ultrasound, or ultrasonic, energy. Ultrasound energy delivery may comprise higher-frequency sound waves that are greater than about 40,000 Hertz (Hz), or alternatively lower frequency sound waves comprising frequencies of about 40,000 Hertz or less. The energy produced by the sound waves may penetrate keratinous tissue, with the depth of penetration dependent upon factors including the acoustic density of the sound waves, the frequency of the sound waves, and the composition of the keratinous tissue. The output energy generally will range from milliwatts to watts.

The delivery of the sound waves may be focused, collimated, diffuse, and combinations thereof. The delivery of the sound waves further may be continuous, pulsed, modulated, non-modulated, and combinations thereof. The ultrasound energy usually is delivered through a transducer head. When used on skin, it is usually placed in direct contact with the skin using a coupling medium, one example of which is an aqueous gel.

Examples of suitable ultrasound devices are disclosed in US 2002/0120225 A1, filed by McDaniel on Jun. 8, 2001; U.S. Pat. No. 6,732,744, issued to Olshavsky et al.; and U.S. Pat. No. 6,544,259, issued to Tsaliovich.

II. Hair Growth Regulating Compounds

The methods described in the present invention comprise the step of applying compositions comprising at least one hair growth regulating compound. Non-limiting examples of suitable hair growth regulating compounds include hexamidine, butylated hydroxytoluene (BHT), hexanediol, panthenol and pantothenic acid derivates, butylated hydroxyanisole (BHA), hexyl isobutyrate, menthyl anthranilate, methofuran, 3-butylidenepthalide, cetyl pyridinium chloride, soy extracts, green tea extracts, catechin compounds, phytosterols, ursolic acid, alpha-glucans, beta-glucans, free radical spin traps (“spin traps”) and mixtures thereof. Alternatively, the hair growth regulating compound is selected from the group consisting of hexamidine, butylated hydroxytoluene (BHT), hexanediol, green tea extracts, catechin compounds, panthenol, pantothenic acid derivatives, alpha-glucans, beta-glucans, free radical spin traps, and mixtures thereof. A. Hexamidine

The compositions of the present invention may comprise an effective amount of hexamidine. “Hexamidine,” as used herein, means the following compound, and includes its isomers, tautomers, salts and derivatives: 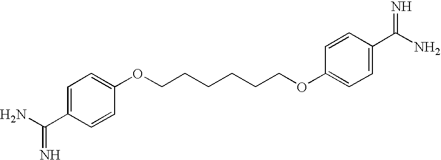
A technical name for the hexamidine of the present invention is 4,4′-(hexamethylenedioxy) dibenzenecarboximidamide. Dermatologically acceptable salts include alkali metal salts, such as sodium and potassium; alkaline earth metal salts, such as calcium and magnesium; non-toxic heavy metal salts; and ammonium and trialkylammonium salts such as trimethylammonium and triethylammonium. Alternatively, the hexamidine is hexamidine isethionate, which is commercially available under the tradename ELASTAB® HP100 from Laboratoires Serobiologiques (Pulnoy, France).

The compositions of the present invention may comprise from about 0.0001% to about 20%, alternatively from about 0.001% to about 10%, alternatively from about 0.01% to about 5%, and alternatively from about 0.1% to about 2% of hexamidine.

B. Butylated Hydroxytoluene (BHT)

The compositions of the present invention may comprise an effective amount of butylated hydroxytoluene (BHT), its isomers, tautomers, salts and derivatives. A technical name for BHT is 2,6-bis(1,1-dimethylethyl)-4-methylphenol. BHT can be purchased from various suppliers, including Eastman Chemical (Kingsport, TN), Alfa Chemical (Kings Point, NY), and Shell Chemical Company (Houston, TX).

The compositions of the present invention may comprise from about 0.0001% to about 50%, alternatively from about 0.001% to about 10%, alternatively from about 0.01% to about 5%, and alternatively from about 0. 1% to about 1% of BHT.

B. Hexanediol

The compositions of the present invention may comprise an effective amount of hexanediol, its isomers, tautomers, salts and derivatives. Some technical names for hexanediol suitable for use herein include 1,6-dihydroxyhexane, 1,6-hexanediol, hexamethylenediol, hexamethylene glycol, and 1,2-hexanediol.

The compositions of the present invention may comprise from about 0.0001% to about 50%, alternatively from about 0.001% to about 10%, alternatively from about 0.01% to about 5%, and alternatively from about 0.1% to about 2% hexanediol.

C. Panthenol and Pantothenic Acid Derivatives

The compositions of the present invention may comprise an effective amount of panthenol and/or pantothenic acid derivatives. Panthenol and pantothenic acid derivatives may include D-panthenol ([R]-2,4-dihydroxy-N-[3-hydroxypropyl)]-3,3-dimethylbutamide), DL-panthenol, pantothenic acids and their salts, preferably the calcium salt, panthenyl triacetate, royal jelly, panthetine, pantotheine, panthenyl ethyl ether, pangamic acid, pantoyl lactose, Vitamin B complex, ethyl panthenol, panthenyl triacetate, and combinations thereof. Alternatively, the panthenol is a d-isomeric forms of a pantothenic acid derivative, and alternatively, the panthenol is d-ethyl panthenol.

The compositions of the present invention may comprise from about 0.01% to 10%, alternatively from 0.1% to about 5%, and alternatively from about 0.2% to about 3% of panthenol.

D. Catechin Compounds

The personal care compositions of the present invention may comprise one or more catechin compounds selected from the group consisting of catechin, epicatechin, epigallocatechin, epicatechin gallate, epigallocatechin gallate, gallocatechin, and mixtures thereof. In one embodiment, the catechin compound is free of caffeine and is extracted and enriched from a green tea plant source. Alternatively, the catechin compound is epigallocatechin gallate. Various purified forms of catechin compounds are commercially available from Sabinsa (Piscataway, NJ), Active Organics (Lewisville, TX), and Arch Personal Care Products (South Plainfield, NJ).

The compositions of the present invention may comprise from about 0.0001% to about 50%, alternatively from about 0.001% to about 10%, alternatively from about 0.01% to about 5%, and alternatively from about 0.1% to about 2.5% of the catechin compound.

E. Glucans

The compositions of the present invention may comprise alpha (a) glucans, beta-(β) glucans, their isomers, salts, derivatives and mixtures thereof. As used herein, “glucans” may refer to alpha-glucans and/or beta-glucans. Examples of alpha- and beta-glucan derivatives include, but are not limited to, sodium carboxymethyl, hydroxypropyl trimonium chloride and palmitate derivatives. Beta-glucans may be linear or branched polymers of glucose and may comprise beta 1-3, beta 1-4 and/or beta 1-6 glycosidic linkages. For example, beta-glucans derived from oats are unbranched linear glucose polymers with about 80% of beta 1-4 glycosidic linkages and about 20% of beta 1-3 glycosilic linkages. Alpha-glucans of the present invention may be linear or branched polymers of glucose with alpha 1-2 and/or alpha 1-3 and/or alpha 1-4 and/or alpha 1-6 glycosidic linkages. For example, alpha-glucans such as alpha-amylose derived from plants are unbranched linear glucose polymers with alpha 1-4 glycosidic linkages and alpha-glucans such as amylopectin are derived from plants are branched glucose polymers with alpha 1-4 glycosidic linkages in the backbone and alpha 1-6 linkages at branch points.

The glucans of the present invention may be derived from natural sources or they may be synthetic in nature. Preferably, the glucans of the present invention are derived from natural sources. Beta-glucans can be isolated from a variety of natural sources, including but not limited to grains, for example oats and barley; the mucilage of plants, for example, mustard; the cell walls of bacteria and yeasts, for example, brewer's yeast; and from fungi. In one embodiment, the compositions of the present invention comprise beta-glucans derived from mustard, oats and mixtures thereof. Alternatively, the beta-glucans are derived from oats. Alpha-glucans can be isolated from a variety of one or more natural sources, including but not limited to, plants, for example potatoes, rice, and corn; animals, for example from liver and muscle tissues; and from micro-organisms, for example from yeast, fungi and bacteria. Beta-glucans are available commercially, for example Drago- Beta-GlucanTM (oat-derived) from Symrise (Totowa, N.J., and Hamburg, Germany), and Mustard Beta-Glucan R25 from Natunola® (Ontario, Canada). Preferred alpha-glucans are available commercially, for example starch, amylase, amylopectin, dextrin, glycogen, and dextran can be obtained from Sigma Chemical Co., St. Louis, Mo.

The compositions of the present invention may comprise alpha and/or beta-glucans varying in average molecular weight of from about 5,000 Da to about 5,000,000 Da. Alternatively, the average molecular weight of the beta-glucans is from about 50,000 Da to about 3,000,000 Da. Alternatively, the average molecular weight is greater than about 3,000,000 Da. Alternatively, the average molecular weight is greater than about 3,000,000 Da.

In one embodiment, the compositions may comprise from about 0.001% to about 20% glucans. Alternatively, the compositions may comprise from about 0.005% to about 10% glucans. Alternatively, the compositions may comprise from about 0.01% to about 5% glucans. Alternatively, the compositions may comprise from about 0.1% to about 1% glucans.

F. Free Radical Spin Traps

The compositions of the present invention comprise one or more spin traps. One non-limiting example free radical spin traps is the class of compounds known as nitrone derivatives, which includes compounds such as a-phenyl butyl nitrone (PBN), a-phenyl butyl nitrone doxylcyclohexane radicals, 5,5-dimethyl pyrroline N-oxide (DMPO), a-(4-pyridyl-1-oxide)-N-tert-butylnitrone (POBN), 2,2,6,6-tetramethyl-piperidine 1-oxide, 4-hydroxytetramethylpiperidine 1-oxide, salts of N-(1-oxido-2,2,6,6-tetramethyl-4-piperidyl)-N,N-dimethyl-N-hydroxyethylammonium, 3,5-dibromo-4-nitroso-benzene-sulfonic acid, 2-methyl-2-nitrosopropane, nitrosodisulfonic acid, a-(4-pyridyl-1-oxide)-N-t-butylnitrone, 3,3,5,5-tetramethylpyrroline N-oxide, and 2,4,6-tri-t-butylnitrosobenzene, N-tert-butyl hydroxylamine, spin-trapping derivatives, salts and isomers of any of the above, and mixtures thereof. In one embodiment, the spin trap is a-phenyl butyl nitrone, N-tert-butyl hydroxylamine, and mixtures thereof.

The compositions of the present invention may comprise from about 0.01% to about 10%, alternatively from about 0.5% to about 7%, and alternatively from about 1% to about 5% of a free radical spin trap.

III. Dermatologically Acceptable Carrier

The personal care compositions of the present invention may comprise a dermatologically acceptable carrier. The dermatologically acceptable carrier may be present in an amount of from about 50% to about 99.99%, alternatively from about 60% to about 99.95%, alternatively from about 70% to about 98%, and alternatively from about 80% to about 95% by weight of the composition.

The carrier can be in a wide variety of forms. Non-limiting examples of emulsions useful herein include oil-in-water, water-in-oil, water-in-silicone, silicone in-water, water-in-oil-in-water, and oil-in-water-in-silicone emulsions. Alternatively, the emulsion is an oil-in-water emulsion. Emulsions also may contain a humectant, such as glycerin, and may contain from about 1% to about 10%, and alternatively from about 2% to about 5%, of a nonionic, anionic or cationic emulsifier. Examples of water-in-silicone and oil-in-water emulsions are described in U.S. Pat. No. 6,238,678, issued to Oblong et al.

IV. Other Ingredients

The compositions of the present invention may contain a variety of other dermatologically-acceptable which are compatible with the energy delivery device. In any embodiment of the present invention, however, the other ingredients useful herein can be categorized by the benefit they provide or by their postulated mode of action. However, it is to be understood that ingredients useful herein can in some instances provide more than one benefit or operate via more than one mode of action. Therefore, classifications herein are made for the sake of convenience and are not intended to limit the active to that particular application or applications listed.

A. Other Hair Growth Regulating Compounds

The compositions of the present invention also may comprise other compounds known in the art to inhibit or otherwise affect hair growth, non-limiting examples of which include natural plant extracts, metabolic modulators, anti-proliferatives, polyamine transport inhibitors, antizyme modulators, signal transduction modifiers, proteases, and protease inhibitors. These and other compounds suitable for use herein are described in U.S. patent applications 2005/0058752 A1, 2005/0065941 A1, 2005/0245615 A1, 2005/0176828 A1, and 2004/0209926 A1.

B. Deodorants

The compositions of the present invention may include deodorants. As used herein, “deodorant” means compounds suitable for regulating malodor, yet which do not provide significant regulation of hair growth. Non-limiting examples of deodorants are found in the class of compounds comprising cyclodextrins. As used herein, the term “cyclodextrin” (CD) comprises any of the known cyclodextrins, such as unsubstituted cyclodextrins capable of forming complexes with odor-causing compounds, and containing from six to twelve glucose units. Suitable cyclodextrins include alpha-, beta-, and gamma-cyclodextrins, their salts, isomers and derivatives, and mixtures thereof. Alpha-, beta-, and gamma-cyclodextrins can be obtained from, among others, American Maize-Products Company (Amaizo), Corn Processing Division, Hammond, Indiana; and Roquette Corporation, Gurnee, Illinois. Many derivatives of cyclodextrins are known, including those disclosed in U.S. Pat. Nos. 3,426,011; 3,453,257; 3,453,258; 3,453,259; 3,453,260; 3,553,191; 3,565,887 issued to Parmerter et al.; 3,459,731, issued to Gramera et al.; 4,535,152, issued to Szejtli et al.; 4,616,008, issued to Hirai et al.; 4,638,058, issued to Brandt et al.; 4,746,734, issued to Tsuchiyama et al.; and 4,678,598, issued to Ogino et al. Examples of cyclodextrin derivatives suitable for use herein are methyl-P-CD, hydroxyethyl-β-CD, and hydroxypropyl-β-CD having varying degrees of substitution, available from Amaizo and from Aldrich Chemical Company, Milwaukee, Wisconsin. Water-soluble derivatives are also highly desirable.

Individual cyclodextrins also can be linked together, for example, by using multifunctional agents to form oligomers, co-oligomers, polymers, copolymers, etc. Examples of such materials are available commerically from Amaizo and from Aldrich Chemical Compnay (β-CD/epichlorohydrin copolymers).

C. Depilatories

Certain embodiments of the present invention may contain a depilatory. As used herein, “depilatory” means an agent capable of removing existing hair, but which does not significantly inhibit hair re-growth and/or regeneration. Without being limited by theory, it is believed that depilatories remove existing hair by cleaving the disulfide bonds in hair keratin, thereby causing the hair fiber to disintegrate. Depilatories useful in the subject invention include ammonium thioglycolate, barium sulfate, calcium thioglycolate, ethanolamine thioglycolate, potassium thioglycolate, sodium thioglycolate, thioglycolic acid, thioacetic acid, and mixtures thereof. Examples of suitable depilatories are described in further detail in U.S. Pat. No. 5,897,857, issued to Hillebrand et al.

D. Particulates

The composition of the present invention may comprise a particulate material. Non-limiting examples of suitable particulate materials can be found in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2728. Other examples of particulate materials useful in the present invention include colored and uncolored pigments, interference pigments, inorganic powders and organic powders other than those described above, composite powders, optical brightener particles, and mixtures thereof. The average size of such particulates in general may be smaller than the aforementioned particulate materials, ranging for example from about 0.1 microns to about 100 microns. These particulates can, for example, be platelet shaped, spherical, elongated or needle-shaped, or irregularly shaped, surface coated or uncoated, porous or non-porous, charged or uncharged, and can be added to the current compositions as a powder or as a pre-dispersion. These particulate materials can be derived from natural and/or synthetic sources.

E. Skin Care Actives

The composition of the present invention may comprise one or more of the following skin care actives: desquamation actives, including but not limited to sulfhydryl compounds, salicylic acid, zwitterionic surfactants, and derivatives and mixtures thereof; anti-acne actives; anti-wrinkle actives and/or anti-atrophy actives, including but not limited to sulfur-containing D and L amino acids, for example, the N-acetyl derivatives such as N-acetyl-L-cysteine and N-acetyl glucosamine; thiols, for example, ethane thiol and glutathione; hydroxy acids; skin peel agents; flavonoids; peptides, for example, peptides derived from soy proteins, palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, and Cu-histidine-glycine-glycine (Cu-HGG, also known as IAMIN®); anti-oxidants, such as Coenzyme Q₁₀ (ubiquinone, vitamin Q₁₀); vitamin B compounds, for example, thiamine (vitamin B 1), pantothenic acid (vitamin B5), L-camitine (vitamin BT), riboflavin (vitamin B2), cobalamine (vitamin B 12), pangamic acid or diisopropylamine dichloroacetate (vitamin B15's); vitamin B₃ compounds; anti-oxidants/radical scavengers, including but not limited to ascorbic acid (vitamin C) and its salts, ascorbyl esters of fatty acids, ascorbic acid derivatives (for example, magnesium ascorbyl phosphate and ascorbyl glucoside), tocopherol (vitamin E), tocopherol sorbate, tocopherol acetate and other esters of tocopherol and idebenone; chelating agents; anti-inflammatory agents, such as corticosteroids, nonsteroidal anti-inflammatory agents (“NSAIDS”), “natural” anti-inflammatory agents, allantoin, bisabolol, and compounds of the licorice plant family (Glycyrrhiza glabra); anti-cellulite agent, for example caffeine, theophylline, theobromine, and aminophylline; topical anaesthetics; skin lightening agents such as kojic acid, niacinamide; ascorbic acid and its derivatives, antimicrobial and/or antifungal actives; sunscreen actives and UV-light absorbers; conditioning agents, such as glycerol, urea, guanidine, and sucrose polyesters; and derivatives and mixtures of any of the above.

E. Substrates

The compositions of the present invention can be applied directly to the skin. Additionally or alternatively, the compositions can be applied with the use of a suitable applicator comprising a substrate material for releasably holding the composition. In one embodiment, the composition is pre-combined with or deposited onto the substrate to form a wipe product, one example of which is a disposable wipe product. Wipe products may be packaged in a relatively dry state, and wetted prior to use, or may be packaged having already been wetted.

Suitable wipe substrates include, but are not limited to, nonwovens, films, foams, sponges, and combinations thereof. Alternatively, the wipe substrates comprise a porous material which is capable of holding the composition within the pores of the substrate. Alternatively, the substrate is nonwoven.

IV. Methods of Use

A. Application of the Compositions

Herein, “compositions” means one or more compositions as described above, uses of which include, but are not limited to, providing a benefit to mammalian keratinous tissue, including regulation of hair growth, bodily malodor, the appearance of hair and/or the appearance of skin. Application of the compositions can occur through a variety of means, including with the fingers or hands, or by using an implement. Non-limiting examples of implements include a pad, cotton ball, applicator pen, spray applicator, substrates and patches. The compositions may be secured to and/or enclosed within the energy delivery device itself. Additionally and/or alternatively, the compositions of the present invention may be administered orally, via an orally acceptable carrier.

Application of the compositions by means of a patch also may be useful for problem skin areas needing more intensive application. The patch can be occlusive, semi-occlusive or non-occlusive and can be adhesive or non-adhesive. The composition can be contained within the patch or be applied to the skin prior to application of the patch. The patch can also include additional actives such as skin conditioners, and chemical initiators for endothermic or exothermic reactions such as those described in U.S. Pat. Nos. 5,821,250, 5,981,547, and 5,972,957 to Wu, et al. The patch may be left on the skin for a period of at least about 5 minutes, alternatively at least about 15 minutes, alternatively at least about 30 minutes, alternatively at least about 1 hour, alternatively overnight as a form of night therapy. The energy may be delivered after removal of the patch, or prior to removal of the patch. When energy is delivered prior to removal of the patch, the patch may be made of a material that is conducive to the form of energy being delivered.

A wide range of quantities of the compositions of the present invention can be employed to improve the condition of the skin. Quantities of the present compositions typically applied per cm² of skin are from about 0.1 mg/cm² to about 20 mg/cm². One useful application amount is about 0.5 mg/cm² to about 10 mg/cm². The composition may be applied to any part of the external portion of keratinous tissue, including face, under-eye area, eyelids, scalp, neck, torso, arms, underarms, hands, legs, feet, eyelashes, eyebrows, and combinations thereof.

B. Delivery of Energy to the Skin

The energy delivery device may deliver energy in a variety of forms, including but not limited to energy in the form of light, heat, sound (including ultrasonic waves), magnetic energy, electromagnetic energy (including radiofrequency waves and microwaves), and combinations thereof. Delivery of energy to the skin means that an effective amount of energy is applied to the keratinous tissue via a means suitable for the type of energy being delivered. The energy from these various sources may be continuous, pulsed, modulated, non-modulated, and combinations thereof. In one embodiment, the energy delivery device is hand-held. Alternatively, the energy delivery device is cordless.

The energy may be applied by holding the device to a single area of keratinous tissue, and subsequently moving the device to another area of tissue (or “stamping”). Alternatively, the energy may be applied as the device is continuously moved, or scanned, across the surface of the tissue. The scanning velocity will depend upon a variety of factors, examples of which include the size of the device, the amount of hair, the type of keratinous tissue, and the amount of energy delivered. The device may be held in substantially continuous contact with the surface of the keratinous tissue, as with laser devices, or may be held at a short distance from the keratinous tissue with the energy directed toward the surface, as with flash lamps.

In one embodiment, the energy output is non-modulated, with said non-modulation regulated by feedback received by the device. This feedback may result from energy being delivered into the skin, where a change is induced in the skin itself or in some compound applied to the skin. Alternatively, the area of the skin in which a change is induced is similar in size to the area to which the energy delivery device delivers energy to the skin. Alternatively, the area of the skin in which a change is induced is larger than the area to which the energy is delivered to the skin.

A temperature change may be simultaneously induced in the keratinous tissue or alternatively, in a compound applied to the surface of the skin. This temperature change is in addition to any temperature change induced by the delivered energy itself. For example, the keratinous tissue may be heated prior to delivery of energy, or alternatively, the keratinous tissue may be cooled after delivery of energy.

For energy derived from ultraviolet light sources, the wavelength will generally fall within the UV-A range, from about 315-400 nm, where “nm” means 1×10⁻⁹ meters. For energy derived from visible light sources, the wavelength will generally range from about 400 nm to about 700 nm. For energy derived from infrared (IR) light sources, the wavelength will generally range from about 700 nm to about to about 3000 nm. The amount of energy delivered, or “output fluence,” may be in the range of about 1 J/cm² to about 100 J/cm², where “J” means Joules. For pulsed light sources, the pulse length may range from about 0.001 seconds to about 3 seconds, with an average pulse duration of from about 0.001 seconds to about 1 second. The surface area of keratinous tissue to be covered will vary depending on the application. These and other parameters relevant to delivery of energy depend upon the type of treatment and the type of tissue to be treated, and will appropriately be selected by one of skill in the art.

C. Application Regimens

The compositions of the present invention may be applied to the skin simultaneously and/or sequentially with delivery of energy to the skin. Alternatively, the energy is delivered prior to application of the composition to the skin. Alternatively, the application regimen may comprise the step of treating the skin with an acute hair growth technology.

One example of a suitable treatment regimen comprises a first time interval, during which delivery of energy occurs to an area of keratinous tissue, followed by a second time interval, or rest period, during which no energy is delivered, followed by a third time interval, during which a composition as described herein is applied to the area of keratinous tissue to which energy was delivered. The duration of the rest period will vary according to the judgment of one of skill in the art. In one embodiment, the rest period lasts about 30 minutes, alternatively from about 20 minutes to about 30 minutes, alternatively at least 30 minutes, alternatively at least one day, and alternatively at least 14 days.

Alternatively, the treatment regimen may comprise a first time interval, during which a composition as described herein is applied, followed by a second time interval, or rest period, during which no energy is delivered, followed by a third time interval, during which energy is applied to the area of keratinous tissue to which the composition was applied.

D. Kit

The present invention further may comprise a kit. The kit may comprise a composition comprising at least one hair growth regulating compound; an energy delivery device; a communication describing one or more of the following: use of the device, applying the composition, complying with a suitable application regimen, use of a coverage indicator; and combinations thereof. The kit further may comprise an outer packaging unit, which in turn may comprise one or more smaller, inner packaging units. The inner packaging units may comprise one or more of the individual components of the kit. The inner and outer packaging units may be of any type suitable for containing, presenting and/or reasonably protecting from damage the contents of the kit. The kit may contain individual inner packaging units, each containing a quantity of composition suitable for use in a single application regimen. In one example, the individual packaging units will contain 10 ml, alternatively 5 ml, alternatively 1 ml, and alternatively 0.5 ml of a composition described herein.

The communication may be printed material attached directly or indirectly to packaging that contains the article of manufacture. Alternatively, the communication may be placed directly or indirectly near a container. Alternatively, the communication may be an electronic or a broadcast message that is associated with the article of manufacture. Alternatively, the communication may describe at least one possible use, capability, distinguishing feature and/or limitation of the article of manufacture.

### EXAMPLES

The following are examples of compositions that may be applied simultaneously and/or sequentially with delivery of energy to the skin.

Content in formulation (g component per 100 g formulation)

| Component | A | B | C | D | E | F |
|---|---|---|---|---|---|---|
| Disodium EDTA | 0.100 | 0.100 | 0.100 | 0.100 | 0.100 | 0.100 |
| Glucan | 1.000 | 1.000 | 1.000 | 1.000 | 1.000 | 1.000 |
| Niacinamide | 0.010 | 4.000 | 1.000 | 3.000 | 2.000 | 0.100 |
| □-phenylbutyl nitrone | 5.000 | 0.01 | 1.00 | 0.05 | 0.1 | 2.00 |
| N-acetyl glucosamine | 0.000 | 2.000 | 0.000 | 0.000 | 2.000 | 0.000 |
| Palmitoyl-pentapeptide (pal-KTTKS) | 0.000 | 0.000 | 0.0003 | 0.000 | 0.000 | 0.000 |
| Phytosterol | 0.000 | 0.000 | 0.000 | 5.000 | 0.000 | 0.000 |
| Ascorbyl glucoside | 0.000 | 0.000 | 0.000 | 0.000 | 2.000 | 0.000 |
| Glucosyl hesperidin | 0.000 | 0.000 | 0.000 | 0.000 | 0.000 | 1.000 |
| Pyridoxine hydrochloride | 0.000 | 0.000 | 0.000 | 0.000 | 0.000 | 0.500 |
| Isohexadecane | 3.000 | 3.000 | 3.000 | 3.000 | 3.000 | 3.000 |
| Isopropyl isostearate | 1.330 | 1.330 | 1.330 | 1.330 | 1.330 | 1.330 |
| Isopropyl N-laurosylsarcosinate | 0 | 0 | 0 | 6.000 | 0 | 0 |
| Sucrose polycottonseedate | 0.670 | 0.670 | 0.670 | 0.670 | 0.670 | 0.670 |
| Polymethylsilsesquioxane | 0.250 | 0.250 | 0.250 | 0.250 | 0.250 | 0.250 |
| Cetearyl glucoside + cetearyl alcohol | 0.200 | 0.200 | 0.200 | 0.200 | 0.200 | 0.200 |
| Behenyl alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerin | 7.000 | 7.000 | 7.000 | 7.000 | 7.000 | 7.000 |
| Titanium dioxide | 0.604 | 0.604 | 0.604 | 0.604 | 0.604 | 0.604 |
| Polyacrylamide + C13-14 isoparaffin + laureth-7 | 3.000 | 2.000 | 2.000 | 2.000 | 2.000 | 2.000 |
| Panthenol | 1.000 | 1.000 | 1.000 | 1.000 | 1.000 | 1.000 |
| Benzyl alcohol | 0.400 | 0.400 | 0.400 | 0.400 | 0.400 | 0.400 |
| Dimethicone + dimethiconol | 2.000 | 2.000 | 2.000 | 2.000 | 2.000 | 2.000 |
| Water (to 100 g) | to 100 | to 100 | to 100 | to 100 | to 100 | to 100 |
| TOTAL | 100 | 100 | 100 | 100 | 100 | 100 |
| Component | G | H | I | J | K | L |
| Disodium EDTA | 0.100 | 0.100 | 0.100 | 0.100 | 0.100 | 0.100 |
| Glucan | 5.000 | 3.000 | 0.1000 | 0.010 | 1.000 | 1.000 |
| Niacinamide | 4.000 | 4.000 | 4.000 | 4.000 | 4.000 | 4.000 |
| □-phenylbutyl nitrone | 5.000 | 5.000 | 5.000 | 5.000 | 5.000 | 5.000 |
| N-acetyl glucosamine | 0.000 | 2.000 | 0.000 | 0.000 | 2.000 | 0.000 |
| Palmitoyl-pentapeptide (pal-KTTKS) | 0.000 | 0.000 | 0.0003 | 0.000 | 0.000 | 0.000 |
| Phytosterol | 0.000 | 0.000 | 0.000 | 5.000 | 0.000 | 0.000 |
| Ascorbyl glucoside | 0.000 | 0.000 | 0.000 | 0.000 | 2.000 | 0.000 |
| Glucosyl hesperidin | 0.000 | 0.000 | 0.000 | 0.000 | 0.000 | 1.000 |
| Pyridoxine hydrochloride | 0.000 | 0.000 | 0.000 | 0.000 | 0.000 | 0.500 |
| Isohexadecane | 3.000 | 3.000 | 3.000 | 3.000 | 3.000 | 3.000 |
| Isopropyl isostearate | 1.330 | 1.330 | 1.330 | 1.330 | 1.330 | 1.330 |
| Isopropyl N-laurosylsarcosinate | 0 | 0 | 0 | 6.000 | 0 | 0 |
| Sucrose polycottonseedate | 0.670 | 0.670 | 0.670 | 0.670 | 0.670 | 0.670 |
| Polymethylsilsesquioxane | 0.250 | 0.250 | 0.250 | 0.250 | 0.250 | 0.250 |
| Cetearyl glucoside + cetearyl alcohol | 0.200 | 0.200 | 0.200 | 0.200 | 0.200 | 0.200 |
| Behenyl alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerin | 7.000 | 7.000 | 7.000 | 7.000 | 7.000 | 7.000 |
| Titanium dioxide | 0.604 | 0.604 | 0.604 | 0.604 | 0.604 | 0.604 |
| Polyacrylamide + C13-14 isoparaffin + laureth-7 | 3.000 | 2.000 | 2.000 | 2.000 | 2.000 | 2.000 |
| Panthenol | 1.000 | 1.000 | 1.000 | 1.000 | 1.000 | 1.000 |
| Benzyl alcohol | 0.400 | 0.400 | 0.400 | 0.400 | 0.400 | 0.400 |
| Dimethicone + dimethiconol | 2.000 | 2.000 | 2.000 | 2.000 | 2.000 | 2.000 |
| Water (to 100 g) | to 100 | to 100 | to 100 | to 100 | to 100 | to 100 |
| TOTAL | 100 | 100 | 100 | 100 | 100 | 100 |

| Component | M | N | O | P |
|---|---|---|---|---|
| AlZr Trichlorohydrex Glycinate | 25 | 25 | 25 | 25 |
| (solid) | | | | |
| Dimethicone (10 cs) | 5.0 | 5.0 | 5.0 | 5.0 |
| Fully Hydrogenated High Erucic | 5.0 | 5.0 | 5.0 | 5.0 |
| Acid Rapeseed oil (HEAR oil) | | | | |
| Hexamidine | 0.1 | 0.3 | 0.1 | 0.3 |
| α-phenylbutyl nitrone | 2 | 2 | 5 | 5 |
| C-18-36 Acid Triglyceride | 1.25 | 1.25 | 1.25 | 1.25 |
| Syncrowax HGLC | | | | |
| Perfume | 0.8 | 0.8 | 0.8 | 0.8 |
| Calcium Pantothenate (solid) | 0.5 | 0 | 3.5 | 0 |
| BHT | 0.5 | 0.5 | 0.5 | 0.5 |
| Tocopherol Acetate | 0.5 | 0 | 0.5 | 0.5 |
| Cyclopentasiloxane | qs | qs | qs | qs |

### Example 1

An effective amount of energy is applied to the skin other than the scalp by means of a laser device. Approximately 0.5 mg/cm² of any one of compositions A-L is applied to essentially the same area of skin. The rate of growth, color and texture of the hair is observed. If necessary, the treatment is repeated.

### Example 2

Approximately 0.5 mg/cm² of any one of compositions A-L is applied to the skin other than the scalp. After 30 minutes, an effective amount of energy is applied by means of a heat delivery device. The treatment regimen may be repeated as necessary.

### Example 3

Approximately 5 mg of any one of compositions A-L is applied to the hair on the scalp. After 5 minutes, energy is delivered by means of an ultrasonic energy delivery device. The hair is rinsed. The treatment regimen may be repeated as necessary.

### Example 4

The skin on the face and neck is pre-heated by means of a heating device. A desired amount of any one of the compositions A-L is applied to the skin on the face and neck. Simultaneously with application of the composition, energy is delivered by means of an ultrasonic energy delivery device. The composition is allowed to remain on the skin.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for providing a benefit to mammalian keratinous tissue, comprising:
a) applying a composition to the keratinous tissue, said composition comprising one or more hair growth regulating compounds; and
b) delivering energy to the keratinous tissue by means of an energy delivery device.

2. The method of claim 1, further comprising at least one additional ingredient, selected from the group consisting of glucans, free radical spin traps, deodorants, depilatories, particulates, desquamation actives, anti-acne actives, anti-wrinkle actives, anti-atrophy actives, anti-oxidant actives, radical scavengers, chelators, anti-inflammatory agents, anti-cellulite agents, topical anesthetics, skin lightening agents, antimicrobial actives, antifungal actives, sunscreens, UV-absorbing agents, conditioning agents, thickening agents, and mixtures thereof.

3. The method of claim 1, wherein said hair growth regulating compound is selected from the group consisting of hexamidine, butylated hydroxytoluene, hexanediol, panthenol and pantothenic acid derivates, butylated hydroxyanisole, hexyl isobutyrate, menthyl anthranilate, methofuran, 3-butylidenepthalide, cetyl pyridinium chloride, soy extracts, green tea extracts, catechin compounds, phytosterols, glycerrhetinic acid, salicylates, ursolic acid, polyamine transport inhibitors, antizyme modulators, and mixtures thereof.

4. The method of claim 1, wherein said energy is in a form selected from the group consisting of light, heat, ultrasonic waves, electrical energy, magnetic energy, electromagnetic energy, radio frequency waves, microwaves, and mixtures thereof.

5. The method of claim 4, wherein said energy is in a form selected from the group consisting of light, heat, ultrasonic waves, radio frequency waves, and mixtures thereof.

6. The method of claim 5, wherein said energy is the form of light energy.

7. The method of claim 6, wherein said light energy is laser-generated light energy.

8. The method of claim 7, wherein said laser-generated light energy is pulsed.

9. The method of claim 7, wherein said laser-generated light energy is non-modulated.

10. The method of claim 1, wherein said energy delivery device is hand-held.

11. The method of claim 10, wherein said energy delivery device is cordless.

12. The method of claim 1, wherein said composition and said energy are applied simultaneously.

13. The method of claim 1, wherein said composition and said energy are applied sequentially.

14. The method of claim 1, wherein said composition is applied to a substrate.

15. The method of claim 2, wherein said glucan is selected from the group consisting of alpha-glucans, beta-glucans, and mixtures thereof.

16. The method of claim 2, wherein said free radical spin trap is selected from the group consisting of a-phenyl butyl nitrone, a-phenyl butyl nitrone doxylcyclohexane radicals, 5,5-dimethyl pyrroline N-oxide, a-(4-pyridyl 1-oxide)-N-tert-butylnitrone, 2,2,6,6-tetramethylpiperidine 1-oxide, 4-hydroxytetramethylpiperidine 1-oxide, and the salts of N-(1-oxido-2,2,6,6-tetramethyl-4-piperidyl)-N,N-dimethyl-N-hydroxyethylammonium, 3,5-dibromo-4-nitrosobenzenesulfonic acid, 2-methyl-2-nitrosopropane, nitrosodisulfonic acid, a-(4-pyridyl-1-oxide)-N-t-butylnitrone, 3,3,5,5-tetramethylpyrroline N-oxide, 2,4,6-tri-t-butylnitrosobenzene, spin-trapping derivatives thereof, and mixtures thereof.

17. The method of claim 2, wherein said skin care active is selected from the group consisting of niacinamide, vitamin E compounds, vitamin C compounds, N-acetyl-D-glucosamine, idebenone, tetrahydrocurcumin, soy proteins, palmitoyl-lysine-threonine, palmitoyl-lysine-threonine-threonine-lysine-serine, and mixtures thereof.

18. The method of claim 1, wherein the benefit is improvement of the appearance of mammalian skin.

19. The method of claim 1, wherein the benefit is regulation of hair growth.

20. The method of claim 1, wherein the benefit is regulating bodily malodor.

21. The method of claim 1, wherein the benefit is regulation of hair appearance.

22. A kit for providing a benefit to mammalian keratinous tissue, comprising:
a) a composition comprising one or more hair growth regulating compounds; and
b) an energy delivery device.

23. The kit of claim 22, further comprising a communication describing a use of the device, a use of the composition, instructions for complying with a suitable application regimen, a use of a coverage indicator; and combinations thereof.

24. An application regimen for providing a benefit to mammalian keratinous tissue, comprising:
a) a first time interval, during which delivery of energy to an area of keratinous tissue occurs;
b) a second time interval, during which no energy is delivered; and
c) a third time interval during which a composition comprising one or more hair growth regulating compounds is applied to said area of keratinous tissue.

25. The application regimen of claim 24, wherein said second time interval comprises from about 20 minutes to about 30 minutes.

26. The application regimen of claim 24, wherein said second time interval is greater than about 30 minutes.

27. The application regimen of claim 24, wherein said second time interval occurs immediately after said first time interval and immediately prior to said third time interval.

28. The application regimen of claim 24, wherein said second time interval occurs immediately after said third time interval and immediately prior to said first time interval.
